(19) United States
(12) Patent Application Publication
(10) Pub. No.: US 2019/0015228 A1
(43) Pub. Date: Jan. 17, 2019

(54) IMPLANTABLE PROSTHESIS FOR THORACIC AORTIC DISEASE INVOLVING AORTIC VALVE DYSFUNCTION

(71) Applicant: Cardiatis S.A., Isnes (BE)

(72) Inventors: Noureddine FRID, Beersel (BE); Edward Bronson DIETHRICH, Phoenix, AZ (US)

(21) Appl. No.: 16/069,034

(22) PCT Filed: Jan. 12, 2017
(86) PCT No.: PCT/EP2017/050568

§ 371 Date: Jul. 10, 2018

(30) Foreign Application Priority Data
Jan. 14, 2016 (EP) 16151194.4

(51) Int. Cl. A61F 2/90; A61F 2/24

(52) U.S. Cl. CPC A61F 2/90; A61F 2/2418; A61F 2250/001; A61F 2230/0006; A61F 2210/0076; A61F 2230/0069

(57) ABSTRACT

The invention relates to an implantable endoluminal prosthesis and a method of using such devices for treatment of a valve dysfunction involving ascending aneurysm. The prosthesis is designed for deployment from the aortic annulus to the aorta. It comprises a self-expandable braided framework able to expand from a radially compressed state in a delivery configuration to a radially expanded state. This framework is formed of braided wires and has a proximal end configured to extend toward the heart and a distal end configured to extent toward away from the heart. The self-expandable braided framework extends along an axis. The framework has a main tubular body of cylindrical form of circular cross-section and at a distal end, a neck the diameter of which is smaller than the one of the self-expandable braided framework, and a transition portion extending between the proximal end of the main tubular body and the distal end of the neck. The main tubular body, said neck and said transition portion form an integrated structure devoid of any impermeable cover layer. The prosthesis further is fitted with a radially collapsible valve body made out of an impermeable material placed within the lumen of the neck.

### FIELD OF THE INVENTION

The present invention relates to implantable endoluminal prostheses. More particularly, it relates to an endoluminal prosthesis for treatment of a thoracic aortic disease, such as aneurysm and dissection of the root and/or ascending aorta. Even more particularly it relates to an endoluminal prosthesis for treatment of a thoracic aortic disease involving cardiac valve dysfunction such as aortic valve regurgitation or aortic valve stenosis.

### BACKGROUND OF THE INVENTION

Thoracic aneurysms and dissections involve one or more aortic segments such as aortic root, ascending aorta, arch and descending aorta, and are classified accordingly. Sixty percent of thoracic aortic aneurysms involve the aortic root and/or ascending aorta, 40% involve the descending aorta, 10% involve the arch, and 10% involve the thoracoabdominal aorta.

Dilatation of the ascending aorta (i.e., ascending aortic aneurysm 40) illustrated in FIG. 1 is known as a common cause of aortic regurgitation because the ascending aneurysm grows not only in diameter but also in length. Such elongation may cause aortic valve 46 incompetence by dislocation of the aortic valve plane towards the left ventricle (arrow 41) and subsequent valve dislocation, causing leaflet prolapse.

Treatment of ascending aortic aneurysm 40 usually requires open surgical repair implying cardiopulmonary bypass (there is no “off-the-pump” option), and generally resecting the aneurysm 40 and replacing the vessel with a prosthetic Dacron tube graft 48 of appropriate size as shown in FIG. 2.

When the aneurysm 40 involves the aortic root and is associated with significant aortic regurgitation, one usually performs a composite aortic repair (Bentall procedure) by using a tube graft 48 with a prosthetic aortic valve sewn to one end. The valve and graft are sewn directly to the aortic annulus 42 and the coronary arteries 44 are then reimplanted into the Dacron aortic graft 48 as illustrated in FIG. 3.

Endovascular repair is also known as a relatively new and minimally invasive technique for treatment of abdominal aortic aneurysm. It delivers an impermeable tube (graft) supported with metallic or plastic frame (stent) via a remote vessel. However, because of its impermeability, this technique cannot be applied to ascending aneurysm repair in which the aneurysm involves important branches (e.g. the coronary arteries 44 and the supra aortic branches 37), otherwise it causes fatal complications with occlusion of the branches.

A new type of aneurysm repair system with a multilayer braided stent (MBS, 49) described in U.S. Pat. Nos. 7,588,597 and 8,192,484 was recently introduced by Frid et al. The repair system comprises a bare (i.e. devoid of any impermeable cover layer) self-expandable metal stent 49 in a straight configuration. MBS consists of a plurality of interconnected layers (i.e. multilayer structure) formed by braiding a plurality of wires. A lattice is defined by the interconnected layers and provides the MBS with an optimized porosity. Instead of mechanically/physically keeping out the blood flow from the aneurysm, MBS allows the blood to flow into the aneurysm sac through its multilayer structure, converting an undesired damaging turbulence in the aneurysmal sac into a smooth laminar flow 50 (FIG. 4), which results in excluding the aneurysm by forming a protecting organized thrombus 51 known as layers of Zhan (FIG. 5), while keeping the branches and collaterals patent.

However, a conventional straight multilayer braided stent (MBS) is not suitable to treat the ascending aneurysm 40 because no adequate healthy landing zones 52 for MBS implantation are available. In order to make the protecting organize thrombus 51, the blood flow in the aneurysmal sac should be laminated. If an adequate healthy landing zone 52 at the beginning of the MBS is missing, a gap may occur between the aortic wall and the MBS 49. This lack of sealing allows undesired turbulence 53 formation in the aneurysmal sac, a phenomenon which is called endoleak, resulting in enlargement of the aneurysm with localized stress brought by turbulence 53 as shown in FIG. 6.

### SUMMARY OF THE INVENTION

A first object of the present invention is to provide a device implantable by endovascular approach for treatment of ascending aortic aneurysm.

Another object of the present invention is to provide a device implantable by endovascular approach for treatment of a valve dysfunction involving ascending aortic aneurysm.

Another object of the invention is ensuring a sealing at a proximal end of a cardiac device in order to reduce the risk of aneurysm rupture.

Another object of the invention is ensuring patency of the coronary arteries while treating an ascending aortic aneurysm or a heart valve dysfunction.

Another object of the invention is ensuring a firm support for an artificial heart valve.

It is still another object of the present invention to provide an implantable medical device and a method for improving the perfusion of organs by lamination through the device, such as the heart through coronaries and brain through the brachiocephalic trunk.

The subject of the present invention is defined in the appended independent claims. Preferred embodiment are defined in the depended claims.

A subject of the present invention is an implantable endoluminal prosthesis suitable for deployment from the aortic annulus to the aorta. The prosthesis comprises a self-expandable braided framework able to expand from a radially compressed state in a delivery configuration to a radially expanded state. The self-expandable braided framework is formed of braided wires having a given diameter (Ø₂₅) and having a proximal end configured to extend toward the heart and a distal end configured to extent toward away from the heart. The self-expandable braided framework extends along an axis. The self-expandable braided framework comprises a main tubular body comprising a lumen in a cylindrical form with a circular cross-section and a constant diameter at the distal end of the self-expandable braided framework, a neck comprising a lumen in a cylindrical form with a circular cross-section and a constant diameter smaller than the one of said main tubular body at the proximal end of the self-expandable braided framework, and a transition portion extending between the proximal end of the main tubular body and the distal end of the neck. The main tubular body, said neck and said transition portion consists of an integrated structure being devoid of any impermeable cover layer, and forming a wall having a thickness (T₂₀). The prosthesis further comprises a radially collapsible valve body comprising an impermeable material placed within the lumen of the neck. In the fully expanded state, the total length of the main tubular body and the transition portion is at least 50 mm, preferably at least 100 mm, more preferably at least 150 mm, even more preferably at least 200 mm. The self-expandable braided framework comprises a plurality of layers of wires made of biocompatible material, Each layer forming a mesh, the meshes forming a lattice with a plurality of wires of given layers. The lattice, when observed normal to a wall of the self-expandable braided framework, defines polygonal opening units. Said biocompatible material is preferably selected from the group consisting of titanium, nickel-titanium alloys such as nitinol and Nitinol-DFT®-Platinum, any type of stainless steels, or a cobalt-chromium-nickel alloys such as Phynox®.

According to a preferred embodiment, a ratio (T₂₀/Ø₂₅) of the thickness (T₂₀) of a wall of the self-expandable braided framework to the diameter (Ø₂₅) of wire is higher than 2.0, preferably at least 3.5, more preferably at least 5.5, even more preferably at least 6.5, still even more preferably at least 7.5.

The self-expandable braided framework advantageously comprises less than 150 wires, preferably at least 90 wires and at most 130 wires. Advantageously, the diameter of wire is more than 180 μm, preferably at least 200 μm and at most 220 μm.

Advantageously, the meshes are interlocked forming a lattice with a plurality of wires of given layers, the wires being interlocked in the mesh of at least one of the adjacent layers.

In a fully expanded state, a surface coverage ratio (SCR) of said self-expandable braided framework is preferably at least 25% and at most 50%, preferably at least 30% and at most 40%, more preferably at most 35%.

According to a preferable embodiment, the self-expandable braided framework further comprises a sealing portion between the proximal end of the braided frame work and the neck, the diameter of sealing portion increasing toward the proximal end of the braided framework.

According to another preferable embodiment, the self-expandable braided framework further comprises an enlarged portion between the distal end of the self-expandable braided framework and the main tubular body, the diameter of enlarged portion increasing toward the distal end of the self-expandable braided framework.

Another subject of the present invention relates to the implantable prosthesis described above for use in treatment for cardiac valve dysfunction involving ascending aortic aneurysm, such as aortic valve regurgitation and aortic valve stenosis.

Another subject of the present invention relates to the implantable prosthesis described above for use in improving perfusion of an organ by covering with said implantable endoluminal prosthesis orifices of the coronaries and the supra aortic branches which carries blood to the heart and the brain.

### BRIEF DESCRIPTION OF THE FIGURES

Other particularities and advantages of the invention will be developed hereinafter, reference being made to the appended drawings wherein:

FIG. 1 is a sketch view of an ascending aortic aneurysm involving cardiac valve dysfunction;

FIGS. 2 and 3 are respectively a sketch view and a view in perspective of an ascending aorta partially replaced with artificial graft by open surgical repair;

FIG. 4 is a schematic longitudinal cut view of a laminated blood flow formed in an aneurysm after implantation of a multilayer braided stent;

FIG. 5 is a schematic longitudinal cut view of an organized thrombus formed in an aneurysm after implantation of a conventional straight multilayer braided stent (MBS);

FIG. 6 is a partially cutaway elevation view of an ascending aortic aneurysm involving cardiac valve dysfunction and conventional straight MBS deployed therein;

FIG. 7 is a side view of an implantable endoluminal prosthesis according to the invention placed in the ventricle of the heart and in the ascending aorta, the arch and the descending aorta;

FIG. 8a is a side view of the prosthesis of FIG. 7 in fully expanded state;

FIGS. 8b and 8c are bottom views of the device of FIG. 8a, respectively with closed an open heart valve;

FIG. 9 is a side view of another embodiment of the prosthesis of the invention in fully expanded state;

FIGS. 10a and 10b are perspective views of the tissues forming the valve body;

FIGS. 11 and 12 are side views of other embodiments of the prosthesis of the invention in fully expanded state;

FIG. 13 is a cut view of a detail of another embodiment of the prosthesis of the invention;

FIG. 14 is a cut view of another embodiment of the prosthesis of the invention placed in the ventricle of the heart and in the ascending aorta;

FIG. 15 is a top view of a prosthesis according to the present invention in expanded state;

FIG. 15a is a schematic magnified view of a portion of the endoluminal prosthesis illustrated in FIG. 15.

FIG. 16 is a side view of a tubular body deployed in a curved lumen;

FIGS. 17 and 18 are perspective views of the device of the invention, respectively in straight fully expanded state and in deployed state in a curved lumen;

FIG. 19 is a schematic magnified view of a portion of a wall of an endoluminal prosthesis according to the present invention.

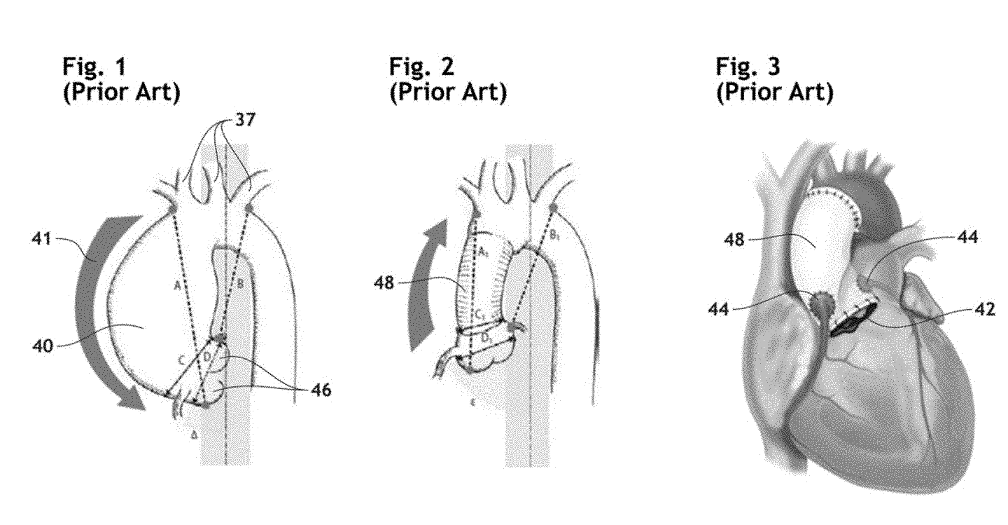

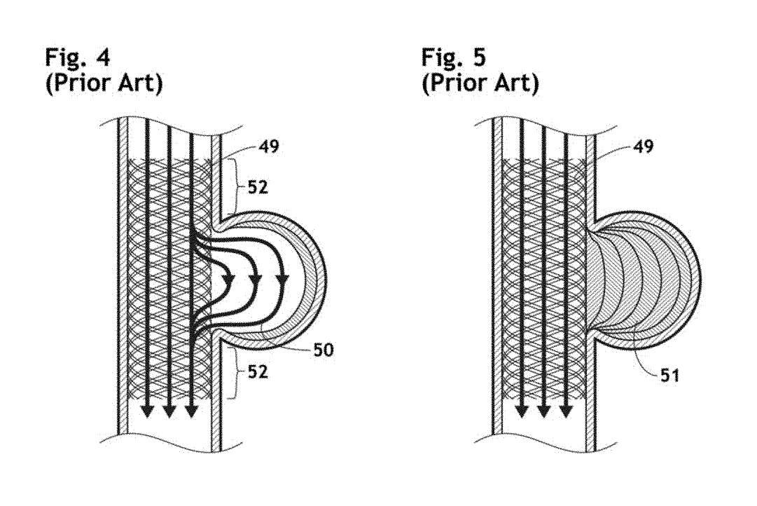

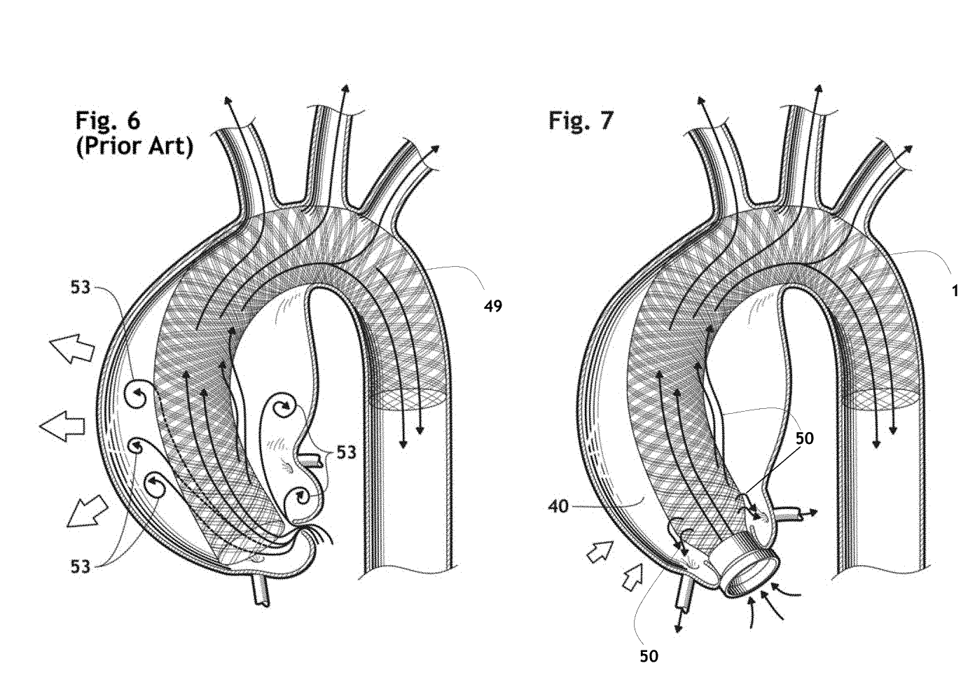

### DETAILED DESCRIPTION OF THE INVENTION

As used hereinafter, the term “implantable” refers to an ability of a medical device to be positioned at a location within a body vessel. Implantable medical device can be configured for transient placement within a body vessel during a medical intervention (e.g., seconds, minutes, hours), or to remain in a body vessel permanently.

The terms “endoluminal” or “transluminal” prosthesis refers to a device adapted for placement in a curved or straight body vessel by procedures wherein the prosthesis is advanced within and through the lumen of a body vessel from a remote location to a target site within the body vessel. In vascular procedures, a medical device can typically be introduced “endovascularly” using a catheter over a wire guide under fluoroscopic guidance. The catheters and wire guides may be introduced through conventional access sites in the vascular system.

The term “catheter” refers to a tube that is inserted into a blood vessel to access the target site. In the present description, a “catheter” will designate either a catheter per se, or a catheter with its accessories, meaning needle, guide wire, introducer sheath and other common suitable medical devices known by the man skilled in the art.

The term “endothelialisation” refers to a cellular process resulting in ingrowth of endothelial cells onto a device.

The term “permanent” refers to a medical device which may be placed in a blood vessel and will remain in the blood vessel for a long period of time (e.g. months, years) and possibly for the remainder of the patient's life.

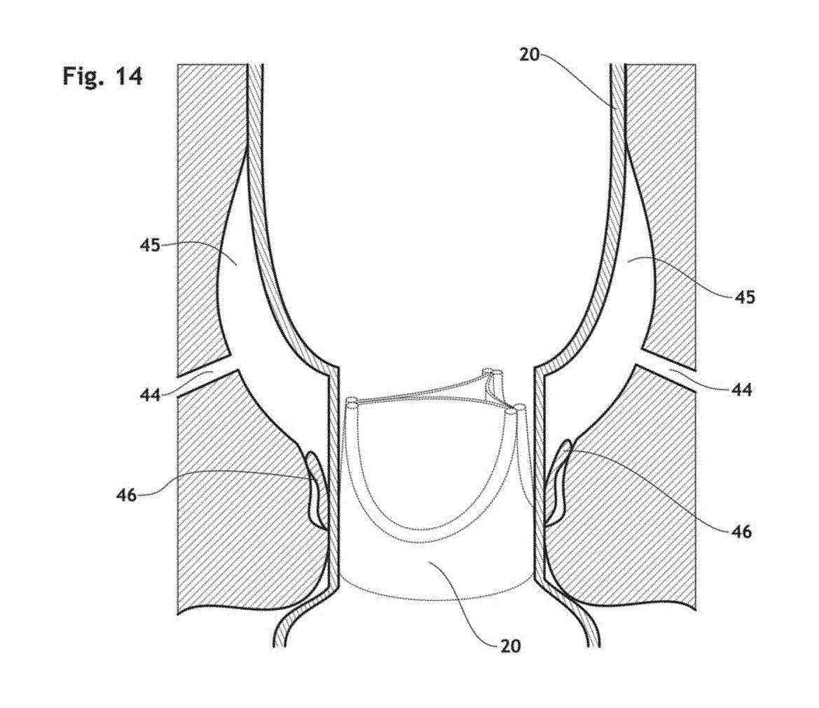

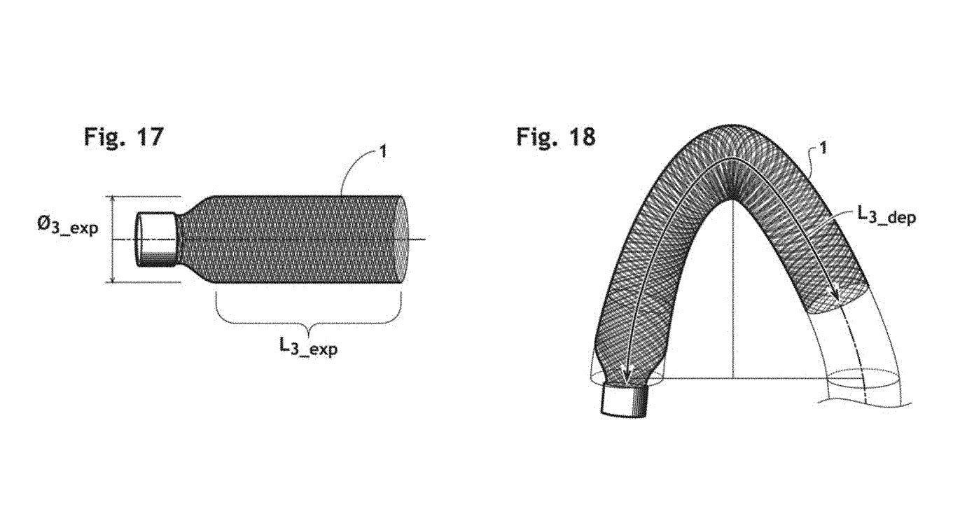

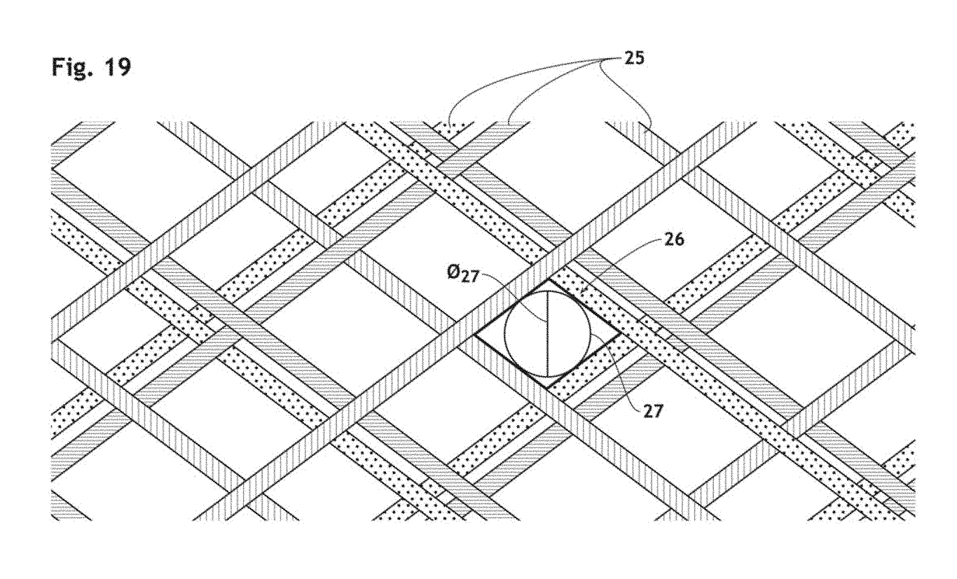

The endoluminal prosthesis 1 is configured to take a compressed shape having a relatively small and relatively uniform diameter when disposed within a delivery system (i.e., “in compressed state”), and to spontaneously take a deployed shape with radially expanded diameter within the delivery location such as a body lumen (i.e., “in deployed state”) as shown in FIGS. 7 and 14. As used herein the terms “expanded shape” or “expanded state” refer to a shape or state resulting from the self-expanding properties of a self-spring-back object (e.g., braided framework 20) when it is allowed to expand without any outer compression force (i.e., non-constricted state) as for example shown in FIGS. 8a to 8c, 9, 5, 11 and 12. Beside these definitions, the term “nominal diameter” designates the diameter of the implantable endoluminal prosthesis when placed in the targeted vessel. Generally, the nominal diameter (Øₙₒᵣ) of a self-expandable device designed to be placed permanently inside a body lumen is 10 to 25% smaller than the external diameter of said device when deployed without external compression force (Øₑₓₚ). Since the diameter (Ø₃₉) of the aorta is generally between 20 mm and 40 mm, the main tubular body 3 of the self-expandable braided framework 20 is accordingly designed and/or manufactured to have a diameter (Ø₃_{_}ₑₓₚ) between 22 mm and 50 mm in expanded state. Variations of the diameter of the prosthesis influence, in turn, its length. As shown in FIGS. 17 and 18, the length (L₃_{_}_{dep}) of the main tubular body 3 of the invention in deployed state is thus larger than its length (L₃_{_}ₑₓₚ) in expanded state. The length-related compression ratio (LCR) of the main tubular body 3 can be defined by the relation:

LCR=(L₃_{_}_{dep}−L₃_{_}ₑₓₚ)/L₃_{_}ₑₓₚ

FIG. 7 represents an implantable endoluminal prosthesis 1 according to the present invention deployed within the aorta, particularly from the aortic annulus 42 to the descending aorta and the arch which covers the coronaries 44 and the supra aortic branches 37.

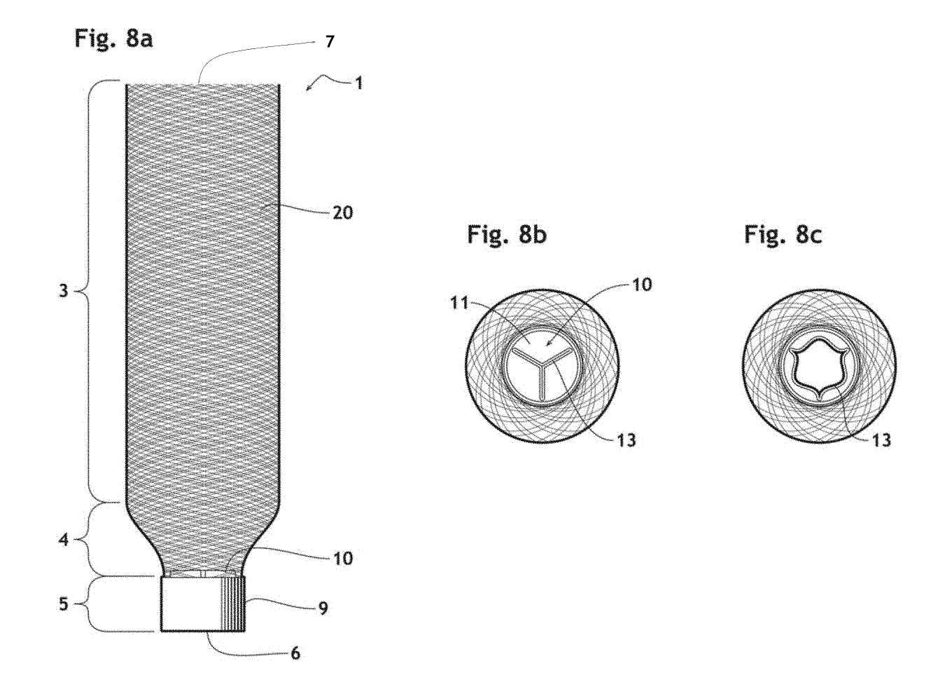

The implantable endoluminal prosthesis 1 according to the present invention comprises a self-expandable braided framework 20 able to expand from a radially compressed state in a delivery configuration to a radially expanded state and a radially collapsible valve body 10 made of an impermeable material, as shown FIGS. 8a to 8c.

The braided framework 20 has a proximal end 6 configured to extend toward the heart and a distal end 7 configured to extent toward away from the heart. The braided framework 20 comprises a main tubular body 3 comprising a lumen in a cylindrical form with a circular cross-section and a constant diameter at the distal end of the braided framework, a neck 5 comprising a lumen of cylindrical form with a circular cross-section and a constant diameter smaller than the one of said main tubular body 3 at the proximal end of the braided framework 20, and a transition portion 4 extending between the proximal end of the main tubular body 3 and the distal end of the neck 5. Said main tubular body 3, said neck 5 and said transition portion 4 consist of an integrated continuous structure made of a multilayer braid and devoid of any impermeable cover layer. The radially collapsible valve body 10 is placed within the lumen of the neck 5. In the fully expanded state, the total length of the main tubular body 3 and the transition portion 4 is at least 50 mm so that the wall of the braided framework 20 completely covers the aneurysm 40, as shown in FIG. 14.

The total length of the main tubular body 3 and the transition portion 4 is, preferably, at least 100 mm in fully expanded state in order to ensure fully covering aneurysmal portion of aorta with the self-expandable braided framework 20. The total length is more preferably at least 150 mm, even more preferably at least 200 mm (still in fully expanded state as shown in FIG. 8), so that the braided framework can have at least 20 mm of healthy landing zone in order to avoid endoleak, which is a main cause of recurrent aneurysms after implantation.

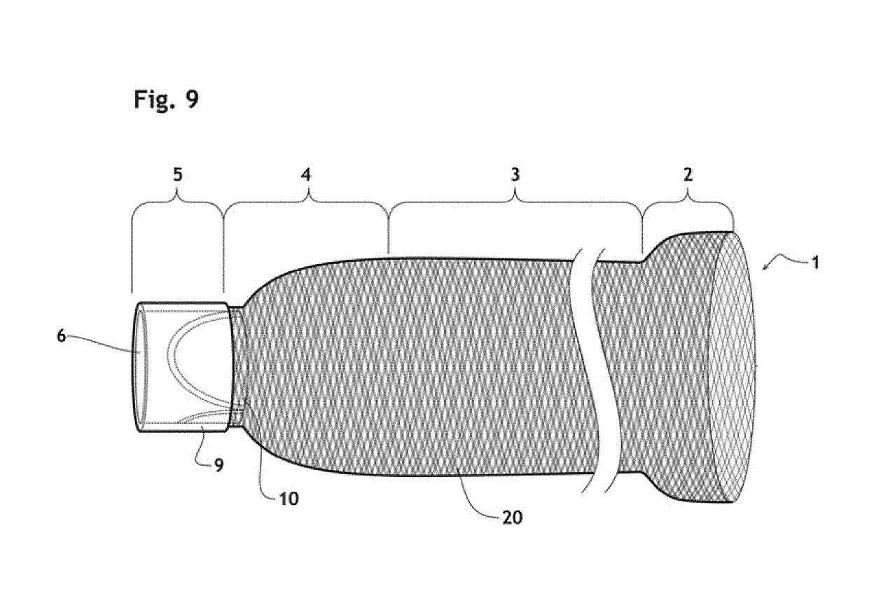

As a preferred embodiment, the self-expandable braided framework 20 further comprises an enlarged portion 2 between the distal end 7 of the braided framework 20 and the main tubular body 3 as illustrated in FIG. 9. The diameter of the enlarged portion 2 increases toward the distal end 7 of the braided framework 20. The enlarged portion 2 also reduce the risk of a device migration and endoleak after implantation.

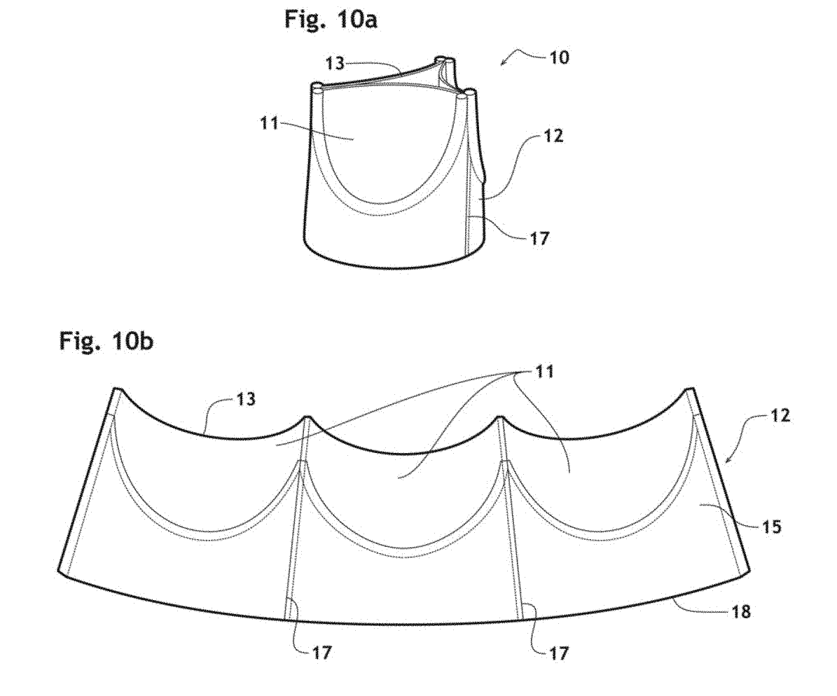

FIGS. 10a and 10b show in a more detailed manner the radially collapsible valve body 10 of the present invention. This valve body comprises a skirt 12 and leaflets 11 which are made of impermeable material. Said skirt 12 and leaflets 11 are preferably cut from a sheet of animal pericardial tissue, such as porcine pericardial tissue, or from another suitable synthetic or polymeric material. The pericardial tissue may be processed in accordance with tissue processing techniques that are per se known in the art of forming and treating tissue valve material. Leaflet 11 has a free edge 13 and a leaflet body 14. Free edge 13 forms coaptation edge 13 of the finished valve body 10. Leaflet body 14 is joined to a skirt 12. Skirt 12 is preferably constructed from the same material as leaflets 11, and comprises concaved portions 15, reinforcing areas 17, and a proximal portion 18. Each concaved portion 15 is joined to a leaflet body 14 of a respective leaflet 11 by sutures or gluing. The valve body 10 is a truncated cone shape having an axis parallel to the one of the braided framework 20 and preferably comprises a reinforcing means, such as overlapped valve body material, metallic wire and plastic bar that are for example affixed to a wall of the skirt 12 between concaved portions 15 along the axis. This prevents the valve body 10 from turning inside out during the cardiac cycle and/or from migration of valve body placed in the braided framework. The proximal portion 18 of skirt 12 is preferably affixed to an inner wall of the proximal end 6 of the braided framework 20 with attaching means such as sutures and gluing.

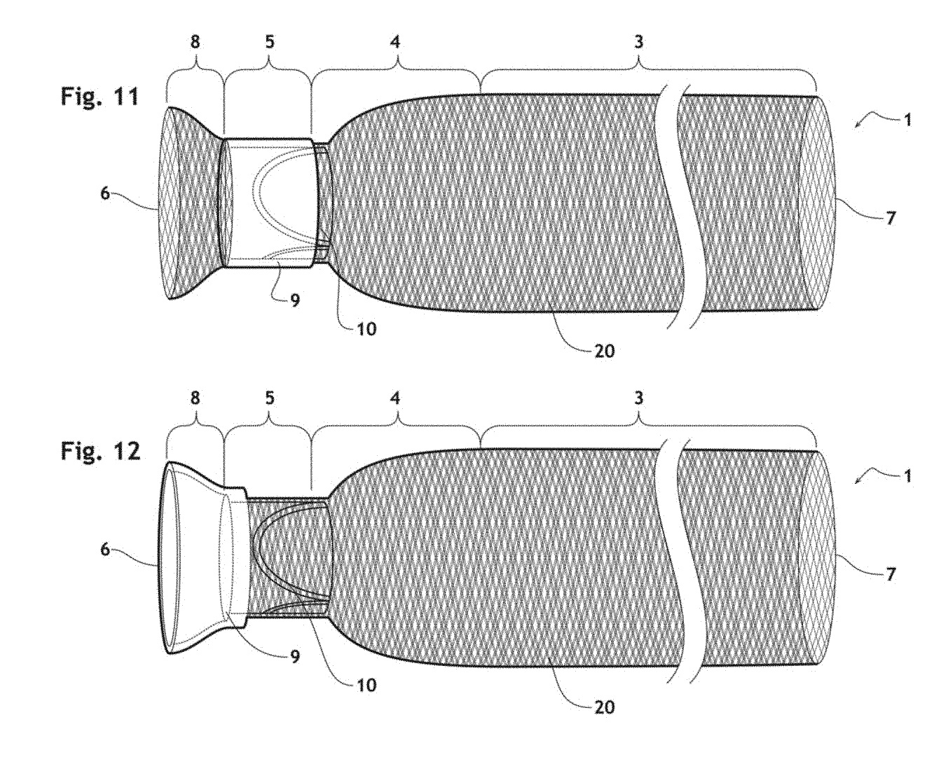

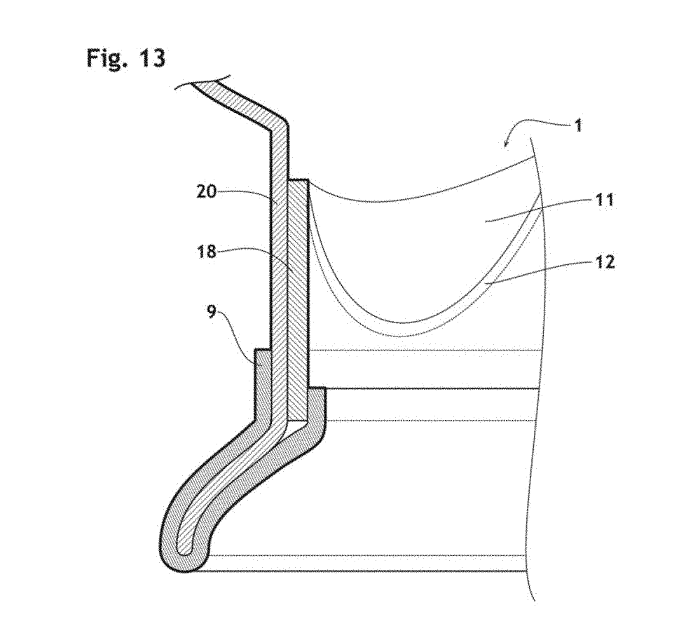

According to another embodiment, illustrated in FIGS. 11 and 12 the self-expandable braided framework 20 further comprises a sealing portion 8 between the proximal end 6 of the braided framework 20 and the neck 5. The diameter of the sealing portion 8 increases toward the proximal end 6 of the braided framework. The sealing portion 8 also reduces the risk of migration of the device away from the valve site after implantation.

In order to ensure sealing of the aneurysm 40 and prevent the blood flow from regurgitation, an impermeable biocompatible sleeve 9 can be used to clamp together both proximal ends, 18 and 6, of skirt 12 and braided framework 20 and affixed by attaching means such as sutures and gluing as illustrated in FIG. 7. This also reduces the risk that wired edges of the proximal end 6 hurt the tissue of aortic annulus 42 when it is deployed in the body. Preferably, the impermeable biocompatible sheet 9 is elastic to accommodate to the change in the length and diameter of the braided framework between its delivery and deployed states.

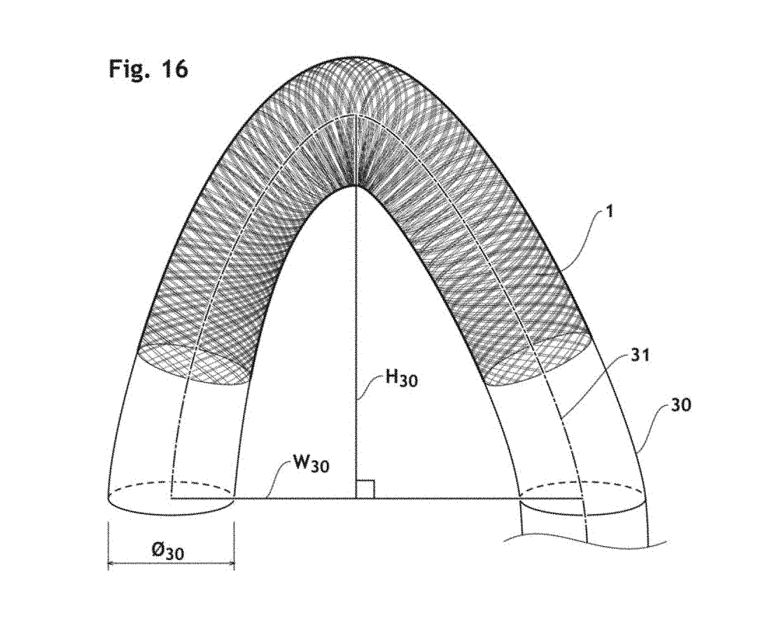

When the tubular body 2 is deployed in a curved lumen 30 as shown in FIG. 16, its length (L₃_{_}_{dep}) is measured along the midpoint 31 of the curve as indicated in FIG. 18.

As depicted in FIG. 19, the braided framework 20 comprises a plurality of layers 22, 23, 24 of wires 25 made of biocompatible material. The wires preferably have a diameter (Ø₂₅) of more than 180 μm, preferably at least 200 μm and at most 220 μm. Each layer of the braided framework 20 forms a mesh. When observed normal with respect to a wall, meshes of the braided frame 20 form a lattices with a plurality of level of wires 25. Preferably, the meshes are interlocked with each other so as to form an interlocked multi-layer structure. The term “interlocked multi-layer” refers to a framework comprising multiple layers, 22, 23, 24, whose plies are not distinct at the time of braiding, for example a given number of wires of the plies 22a of the first layer 22 being interlocked with the plies 23a of the second layer 23 and/or other layers 24. Said interlocked multi-layer, for example, can be formed by using the braiding machine described in EP1248372. The braided framework 20 of the endoluminal prosthesis 1 is made of less than 150 wires 25, preferably at least 90 wires at most 130 wires. The surface coverage ratio (SCR) of the braided framework 20 is defined by the relation:

SCR=S_{w}/Sₜ

wherein: “S_{w}” is the actual surface covered by wires 25 composing the braided framework 20, and “Sₜ” is the total surface area of the wall of the braided framework 20. In a fully expanded state, SCR of the braided framework 20 is preferably at least 25% and at most 50%, preferably at least 30% and at most 40%, more preferably at most 35%.

The curve of the aortic arch 39 is generally defined by measuring the width (W₃₉) and height (H₃₉) of the curve as described by Ou et al. in J. Thrac. Cardiovasc. Surg. 2006; 132: 1105-1111. Width (W₃₉) is measured as the maximal horizontal distance between the midpoints 31 of the ascending and descending aorta 39 close to the axial plane going through the right pulmonary artery (RPA); and height (H₃₉) of the aortic arch is measured maximal vertical distance between (W₃₉) and the highest midpoint 31 of the aortic arch 39 as depicted in FIG. 16. The ratio H₃₉/W₃₉ is generally in a range of 0.5 to 0.9. For example, when the value is 0.9 (the worst scenario), the aortic arch is extremely acute as depicted in FIG. 16. This can cause a kinking of previously described “conventional” stents, which have poor hoop force. Furthermore, one will notice the difference of mesh opening between its straight form greater in comparison with the one deployed in a curve having about 0.6 of the H/W ratio (which is usually observed in healthy aortas). As one of the advantages of the present invention, even if the endoluminal prosthesis 1 is deployed in a C-curved lumen 30 with the H₃₀/W₃₀ ratio between 0.5 and 0.9, the braided framework 20 with a ratio T₁/Ø₂₅ of at least 3.5 (preferably 5.5, more preferably at least 6.5, even more preferably at least 7.5), can provide a surface coverage ratio (SCR) within the desirable range along its outer curve 29, i.e. at least 35%, resulting in maintaining the desired effects at the inlet of supra aortic branches 37 (i.e., laminar effect, improvement of perfusion).

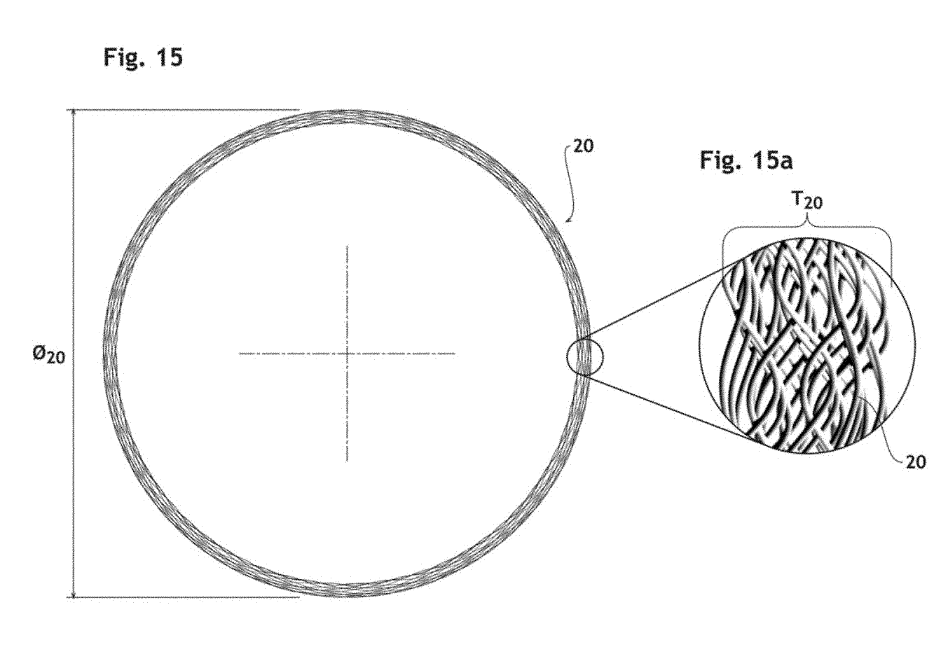

As another advantages of the present invention the braided framework 20, having higher value of the ratio T₂₀/Ø₂₅, can effectively form a thrombus in the aneurysmal sac in comparison with a braided framework having lower T₂₀/Ø₂₅ ratio. The ratio (T₂₀/Ø₂₅) of the wall thickness (T₂₀) of the braided framework 20 to the wire diameter (Ø₂₅) being more than 2.0 characterizes the braided framework having more than a single layer of mesh. The greater the ratio T₂₀/Ø₂₅, the more layers the braided framework 20 will comprise. Each wire forming multiple-layers aligned substantially parallel in the wall, as shown in FIG. 15, works to make the blood flow be laminated which gets through the wall of the endoluminal prosthesis 1.

Furthermore, interlocked multiple-layer configuration having a ratio T₂₀/Ø₂₅ higher than 3.5 brings about an important technical property: when it is deployed in a curved lumen having an H/W ratio between 0.5 and 0.9, the SCR can keep its desirable value, namely at least 25% and at most 50%, even at the outer side of the curve 29 as defined in FIGS. 11 and 14. Since the mouths of the supra aortic branches are located at the outer side of the arch, it is most important to set an optimal opening size at the outer side when deployed in an aortic arch geometry in order to maintain desirable effects provided by the prosthesis. Wires of the interlocked multiple-layer configuration of the invention shift to keep a regular distance between adjacent parallel resulting in that the SCR can stays almost the same either in a curved state or in straight configuration. On the Contrary, when a conventional single-layer mesh-like tube having less than 2.0 of T₂₀/Ø₂₅ is deployed in a curved lumen, the SCR at the outer side of the curve are much lower than the SCR in a straight configuration. Therefore, the ratio T₂₀/Ø₂₅ of the braided framework 20 of the invention should be more than 2.0, preferably at least 3.5, more preferably at least 5.5, even more preferably at least 6.5, still even more preferably 7.5.

Studies and experiments carried by the inventor led to surprising and unexpected conclusions. The perfusion in the branches is improved in accordance with the increase of the ratio T₂₀/Ø₂₅. “Perfusion” is, in physiology, the process of a body delivering blood to capillary bed in its biological tissue. The terms “hypoperfusion” and “hyperperfusion” measure the perfusion level relative to a tissue's current need to meet its metabolic needs. Since the implantable medical device of the invention increases the perfusion in the supra aortic branches it covers, the functioning of the organs to which the supra aortic branches carries the blood is improved. Therefore, the ratio T₂₀/Ø₂₅ of the braided framework 20 of the invention should be more than 2.0, preferably at least 3.5, more preferably at least 5.5, even more preferably at least 6.5, still even more preferably 7.5.

As another surprising effect, against the expectation that a space between an aneurysmal wall and endoluminal prosthesis would be occluded by thrombus, the aneurysm including coronary arteries shrinks directly instead of forming thrombus in the aneurysmal sac while still maintaining the blood flow into the arteries. The inventor assumes that by sealing the beginning of aorta with its valve portion, undesired turbulence 53 are eliminated and desired smooth flow are created in this volume. It accelerates the non-turbulent blood flow entering the branches while decreasing the pressure under Venturi effect, resulting in shrinkage of the aneurysmal sac.

The biocompatible material used in the invention is preferably a metallic substrate selected from a group consisting of stainless steels (e.g., 316, 316L or 304); nickel-titanium alloys including shape memory or superelastic types (e.g., nitinol, Nitinol-DFT®-Platinum); cobalt-chrome alloys (e.g., elgiloy); cobalt-chromium-nickel alloys (e.g., phynox); alloys of cobalt, nickel, chromium and molybdenum (e.g., MP35N or MP20N); cobalt-chromium-vanadium alloys; cobalt-chromium-tungsten alloys; magnesium alloys; titanium alloys (e.g., TiC, TiN); tantalum alloys (e.g., TaC, TaN); L605. Said metallic substrate is preferably selected from the group consisting of titanium, nickel-titanium alloys such as nitinol and Nitinol-DFT®-Platinum, any type of stainless steels, or a cobalt-chromium-nickel alloys such as Phynox®.

Method of Deployment

According to one preferred method, the endoluminal prosthesis 1 of the invention is deployed by using an endoluminal prosthesis delivery apparatus. This apparatus is designed to be driven by an operator from the proximal site on through the vascular system so that the distal end of the apparatus can be brought close to the implantation site, where the prosthesis 1 can be unloaded from the distal end of the apparatus. The delivery apparatus comprises the prosthesis 1 itself, a prosthesis receiving region wherein the prosthesis has been introduced, a central inner shaft and a retracting sheath. Preferably, the apparatus further comprises a self-expanding holding means that is compressed within the sheath, the distal portion of which encircles the proximal potion of the prosthesis, and the proximal end of which is permanently joined to the inner shaft with a joint so as to provide the apparatus with a function of re-sheathing a partially unsheathed prosthesis into a retracting sheath. To deploy the prosthesis 1 at a desired location in the aorta, the distal end of the retracting sheath is brought to the aortic annulus and the retracting sheath is progressively withdrawn from over the prosthesis 1 toward the proximal end of the delivery apparatus. Once the sheath is adjacent the proximal end of the holding means, the prosthesis 1 is partially allowed to self-expand to a deployed shape. By continually retracting the sheath proximally, the holding means is released from the sheath and deploys while under the effect of the temperature of the organism and/or because of their inherent elasticity. In order to prevent a prosthesis migration after implantation, an oversized prosthesis 1 is generally chosen which has a diameter in its “nominal” expanded state being 10-40% greater than the diameter of the body lumen at the implantation site. Such prosthesis 1 exerts a sufficient radial force on an inner wall of the body lumen and is thus fixed firmly where it is implanted. Since, upon deployment, the radial force provided by the deployed part of the prosthesis 1 onto the wall of the aorta becomes greater than the grasping force of the deployed holding means in its deployed state, the holding means can release the prosthesis at the deployed position without undesired longitudinal displacement when retracting the inner shaft proximally together with the sheath.

1. An implantable endoluminal prosthesis suitable for deployment from an aortic annulus to an aorta comprising:
1) a self-expandable braided framework extending along an axis able to expand from a radially compressed state in a delivery configuration to a radially expanded state, the self-expandable braided framework being formed of a plurality of braided wires having a given diameter (Ø₂₅) and having a proximal end configured to extend toward the heart, and a distal end configured to extend away from the heart, the self-expandable braided framework comprising:
a) toward the distal end, a main tubular body comprising a lumen in a cylindrical form with a circular cross-section and a constant diameter;
b) toward the proximal end, a neck comprising a lumen in a cylindrical form with a circular cross-section and a constant diameter smaller than the constant diameter of said main tubular body; and
c) a transition portion extending between the proximal end of the main tubular body and the distal end of the neck,
said main tubular body, said neck and said transition portion including an integrated structure being devoid of an impermeable cover layer, and forming a wall having an average thickness (T₂₀),
2) a radially collapsible valve body comprising an impermeable material placed within the lumen of the neck,
wherein in the radially expanded state, a total length of the main tubular body and the transition portion is at least 50 mm, wherein the plurality of braided wires of the self-expandable braided framework are made of biocompatible material and form a lattice when observed normal to a wall of the self-expandable braided framework, the lattice defining polygonal opening units, a ratio (T₂₀/Ø₂₅) of the average thickness (T₂₀) of a wall of the self-expandable braided framework to the diameter (Ø₂₅) of a wire being greater than 2.0, the self-expandable braided framework comprising less than 150 wires.

2. The implantable endoluminal prosthesis according to claim 1, wherein the braided framework comprises a plurality of layers of the wires, each layer forming a mesh, the meshes are interlocked, the wires being integrated in the mesh of at least one of the adjacent layers.

3. The implantable endoluminal prosthesis according to claim 1, wherein, in the radially expanded state, the total length of the main tubular body and the transition portion is at least 150 mm.

4. The implantable endoluminal prosthesis according to claim 1, wherein the ratio (T₂₀/Ø₂₅) is at least 3.5.

5. The implantable endoluminal prosthesis according to claim 1, wherein the diameter of the wires is larger than 180 μm.

6. The implantable endoluminal prosthesis according to claim 1 wherein, in a fully expanded state, a surface coverage ratio (SCR) of said self-expandable braided framework is at least 25% and at most 50%.

7. The implantable endoluminal prosthesis according to claim 1 wherein, the self-expandable braided framework further comprises a sealing portion between the proximal end of the braided framework and the neck, the diameter of the sealing portion increasing toward the proximal end of the braided framework.

8. The implantable endoluminal prosthesis according to claim 1, wherein the self-expandable braided framework further comprises an enlarged portion between the distal end of the self-expandable braided framework and the main tubular body, the diameter of the enlarged portion increasing toward the distal end of the self-expandable braided framework.

9. The implantable endoluminal prosthesis according to claim 1, wherein the biocompatible material is a metallic substrate selected from the group consisting of titanium, a nickel-titanium alloy, a stainless steel, and a cobalt-chromium-nickel alloy.

10. The implantable endoluminal prosthesis according to claim 1 for use in treatment for cardiac valve dysfunction involving ascending aortic aneurysm.

11. The implantable endoluminal prosthesis for use according to claim 10 wherein the cardiac valve dysfunction is aortic valve regurgitation or aortic valve stenosis.

12. The implantable endoluminal prosthesis according to claim 1 for use in improving perfusion of an organ by covering with said implantable endoluminal prosthesis orifices of the coronaries and the supra aortic branches which carries blood to the heart and the brain.

13. The implantable endoluminal prosthesis according to claim 1, wherein the self-expandable braided framework comprises at least 90 wires and at most 130 wires.

14. The implantable endoluminal prosthesis according to claim 1, wherein the diameter of the wires is at least 200 μm and at most 220 μm.
